(19) United States
(12) Patent Application Publication
(10) Pub. No.: US 2025/0152694 A1
(43) Pub. Date: May 15, 2025

(54) METHODS AND COMPOSITIONS FOR MULTIVALENT VACCINES

(71) Applicant: Board of Regents, The University of Texas System, Austin, TX (US)

(72) Inventors: Zackary Armstrong, San Antonio, TX (US); Karl Klose, San Antonio, TX (US); Xhavit Zogaj, San Antonio, TX (US)

(21) Appl. No.: 18/942,731

(22) Filed: Nov. 10, 2024

Related U.S. Application Data
(60) Provisional application No. 63/598,067, filed on Nov. 10, 2023.
(60) Provisional application No. 63/708,731, filed on Oct. 17, 2024.

(51) Int. Cl. A61K 39/116; A61K 39/00; A61K 39/02; A61K 39/07; C07K 14/24; C07K 14/32; C12N 1/20

(52) U.S. Cl. CPC A61K 39/116; A61K 39/0291; A61K 39/07; C07K 14/24; C07K 14/32; C12N 1/20; A61K 2039/522; A61K 2039/70; C07K 2319/034; C07K 2319/035

(57) ABSTRACT

Certain embodiments are directed to an attenuated bacterial platform for autotransporter-mediated display of antigens. In certain aspects the display is surface display or periplasmic display. In other aspects the antigen is a microbial antigen. The microbial antigen can be a bacterial or viral antigen. The antigen can be antigen derived from a biothreat.

### RELATED APPLICATIONS

This application claims priority to U.S. Provisional Application 63/598,067 filed Nov. 10, 2023 and U.S. Provisional Application 63/708,731 filed Oct. 17, 2024, each of which are incorporated herein by reference in their entirety.

### REFERENCE TO SEQUENCE LISTING

A sequence listing required by 37 CFR 1.821-1.825 is being submitted electronically with this application. The sequence listing is incorporated herein by reference. The sequence listing that is contained in the file named “UTSAP0141US” which is 33 KB (as measured in Microsoft Windows®) and was created on Nov. 10, 2024.

### FIELD

This invention relates generally to the field of medicine and infectious diseases. More particularly, it relates to engineering of vaccine strains of Francisella species and methods of using the same.

### BACKGROUND

Antigen display in bacteria is an aspect of both natural bacterial physiology and biotechnological applications, particularly in vaccine development and immunology research. Some bacteria display antigens on their surface to evade a host's immune system. These antigens often mimic host molecules or are highly variable allowing bacteria like Neisseria gonorrhoeae or Streptococcus pneumoniae to change their surface proteins to avoid recognition by antibodies. Some pathogenic bacteria express antigens as part of their virulence factors. For instance, the flagellin protein in flagella or the lipopolysaccharides (LPS) in the outer membrane of Gram-negative bacteria can act as antigens triggering immune responses.

Bacteria can be engineered to display antigens that can be used as subunit vaccines. These vaccines contain only the antigenic parts of the pathogen, reducing the risk of adverse reactions compared to whole-cell vaccines. The host bacteria is not a component of this type of vaccine. An example is the acellular pertussis vaccine, which includes only selected antigens of Bordetella pertussis. Also, genetically modified bacteria can be used to display antigens from other pathogens (heterologous antigens). For example, Salmonella typhimurium has been engineered to display HIV antigens, providing a live vector vaccine.

Bacterial surface display involves genetically fusing an antigen to a surface protein of a bacterium allowing the antigen to be presented on the bacterial cell surface. This method is useful for screening libraries of antigens for vaccine candidates or for studying protein-protein interactions. Antigens can be anchored to the bacterial surface using various mechanisms. For Gram-negative bacteria like E. coli, proteins like autotransporters, ice nucleation proteins, or outer membrane proteins (e.g., Lpp′OmpA) can be used to anchor antigens. Typically, the gene encoding the antigen is fused to that of a surface protein, ensuring that the antigen is co-expressed and displayed on the bacterial membrane. Ensuring the antigen remains stable and immunogenic on the bacterial surface can be challenging. The immune system might recognize the bacterial components themselves as antigens diluting the response to the displayed antigen.

There remains a need for additional systems, compositions, and methods for producing immunogenic compositions, particularly for biothreats.

### SUMMARY

To address deficiencies in the field of medicine and particularly the area of vaccines and vaccination, the Inventors have developed an attenuated Francisella, e.g., Francisella novicida (F. novicida), vaccine platform. The term “attenuation” or “attenuated” means a pathogen is kept alive but exhibits reduced virulence such that it does not cause the disease caused by the virulent pathogen. The attenuation of a particular strain may result from mutagenesis, deletion or inactivation of targeted genes, or natural attenuation, or a combination thereof. Vaccine platform in the context of the current invention is a live-attenuated bacteria (e.g., a live F. novicida bacteria that is immunogenic but does not pathologically infect a subject) that is used to deliver heterologous antigens. In certain aspects the vaccine platform uses an autotransporter-mediated display for antigens. The vaccine platform can present multiple distinct antigens (multivalent vaccines). In certain aspects an antigen(s) is displayed on the bacterial surface (surface display) or in the periplasm (periplasmic display). In other aspects the antigen can be a microbial antigen, in particular a bacterial antigen(s) (e.g., a bacterial biothreat or biological weapon) or a viral antigen(s) (e.g., SARS-COV-2, or viral biothreat). In particular aspects expression cassettes or constructs can be one or more of the constructs described herein (e.g., schematically represented in FIG. 4B, FIG. 10, FIG. 11, FIG. 14, FIG. 15, FIG. 16, FIG. 17, FIG. 18).

The vaccine platform can be engineered to express selected antigens and/or biothreat antigens. In certain aspects the biothreat antigens are one or more Category A, Category B, and/or Category C antigens. In particular embodiments the biothreat antigen is an antigen from a Category A agent. In a particular aspect the Category A agent is Bacillus anthracis (Anthrax), Clostridium botulinum toxin (Botulism), Yersinia pestis (Plague), Variola major (Smallpox) and other pox viruses, Francisella tularensis (Tularemia), Viral hemorrhagic fevers (Filoviruses like Ebola and Marburg, and Arenaviruses like Lassa), and/or smallpox. In particular aspects the biothreat antigen is from Yersinia pestis (Yp) and/or Bacillus anthracis (Ba) and is expressed in a live-attenuated Tularemia vaccine strain creating a multivalent vaccine platform to protect against multiple biothreats (F. tularensis, Yersinia pestis, and Bacillus anthracis) with a single vaccine platform.

Certain embodiments are directed to a live attenuated Francisella, e.g., Francisella novicida, bacterium expressing a heterologous antigen, the heterologous antigen being operably coupled to a leader sequence or surface protein that localizes the heterologous antigen in the periplasm or on the surface of the live attenuated bacteria. The surface protein can be an autotransporter translocator domain. The autotransporter can be a YfaL autotransporter. The leader sequence can be selected from PelB, PepO, or TorA leader sequence. In certain aspects at least 1, 2, 3, 4, 5 or more heterologous antigens are expressed individually or as a fusion protein. The heterologous antigens expressed as a fusion protein can be separated by a self-cleaving amino acid sequence. The heterologous antigens can be independently translated from a polycistronic mRNA.

Other embodiments are directed to an attenuated F. novicida, bacterium expressing a heterologous antigen, the heterologous antigen being operably coupled to an autotransporter translocator domain. The autotransporter can be a YfaL autotrasnporter.

Certain embodiments are directed to inducing an immune response in a subject to at least 2, 3, 4, 5, or more pathogens by administering a live attenuated vaccine composition described herein. In certain aspects the vaccine composition is administered intranasally, by inhalation, or to the lungs (pulmonary vaccination). The subject can be a human or an animal (livestock or companion animal). In certain aspects the subject is at risk of being infected with a biothreat or other pathogen. Being at risk of infection refers to the state where an individual or a group has an increased likelihood of contracting an infectious disease. This risk can be influenced by various factors: exposure, coming into contact with pathogens through air, physical contact, contaminated surfaces, water, food, or vectors like mosquitoes; immune system status, individuals with weakened immune systems (immunocompromised) due to conditions like HIV/AIDS, undergoing chemotherapy, or having autoimmune disorders are more susceptible; high-risk behaviors or activities, such as travel to areas with high infection rates or where certain diseases are endemic, entering a battlefield or area targeted by bioterrorism; close contact with infected individuals or animals, such as in healthcare settings or within households; or the like. To mitigate being at risk of infection, preventive measures include vaccinations.

Certain embodiments are directed to a nasal delivery system including a vaccine composition described herein.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. KKF768/PelB-RBD (periplasmic) Vaccination with KKF768 expressing PelB-RBD under groELp elicited anti-RBD humoral responses in vaccinated BALB/c mice.

FIG. 2. Neutralization Assay (Neutralization of isolate: USA-WA-1-2020-nLuc) Antisera from KKF768/PelB-RBD-vaccinated mice show neutralization activity against SARSCOV2.

FIG. 3A-3B. Illustration of Autotransporter YfaL. (A) Domain structure. (B) Structural prediction. AIDA-I family E. coli 0157: H7 str. Sakai 3 domains: N-term signal peptide Passenger C-term linker-translocator

FIG. 4A-4B. YfaL Surface Display Design (A) constructs (B) illustration of YfaL structure.

FIG. 5. Detection of SARS-CoV2 S antigens: Wuhan isolate.

FIG. 6. Detection of SARS-CoV2 S antigens: Omicron B.1.1.529 variant. Expression of YfaL- and Periplasmic-SARS2 antigens in KKF768 detectable by western immunoblot. OD₆₀₀=1, Anti-RBD, mAb GeneTex (HL257).

FIG. 7. Detection of YfaL-SARS-CoV2 localization on surface of KKF768.

FIG. 8. Detection of YfaL-RBD localization on surface of KKF768.

FIG. 9. Optimization of autotransporter surface display system: Smaller epitopes. YfaL-FLAG is localized on the surface of KKF768 and does not significantly affect growth.

FIG. 10. Illustrates examples of targeted antigens and leader sequences for periplasmic localization of antigens.

FIG. 11. Illustration of various plasmid configurations and confirmation of expression.

FIG. 12. Western blot detection of recombinant PA (rPA) expression.

FIG. 13. Western blot detection of recombinant LcrV expression.

FIG. 14. Illustration of the E. coli TAT secretion system for periplasmic localization.

FIG. 15. Illustration of various methods for localization of antigens on the surface or in the periplasm.

FIG. 16. Illustration of one example of a trivalent vaccine construct encoding: 1. Two operon expression construct or 2. fusion protein construct.

FIG. 17. Plasmid map for a lcrV/pagA construct comprising a pel1 leader.

FIG. 18. Plasmid map for a lcrV/pagA construct comprising a pelB leader.

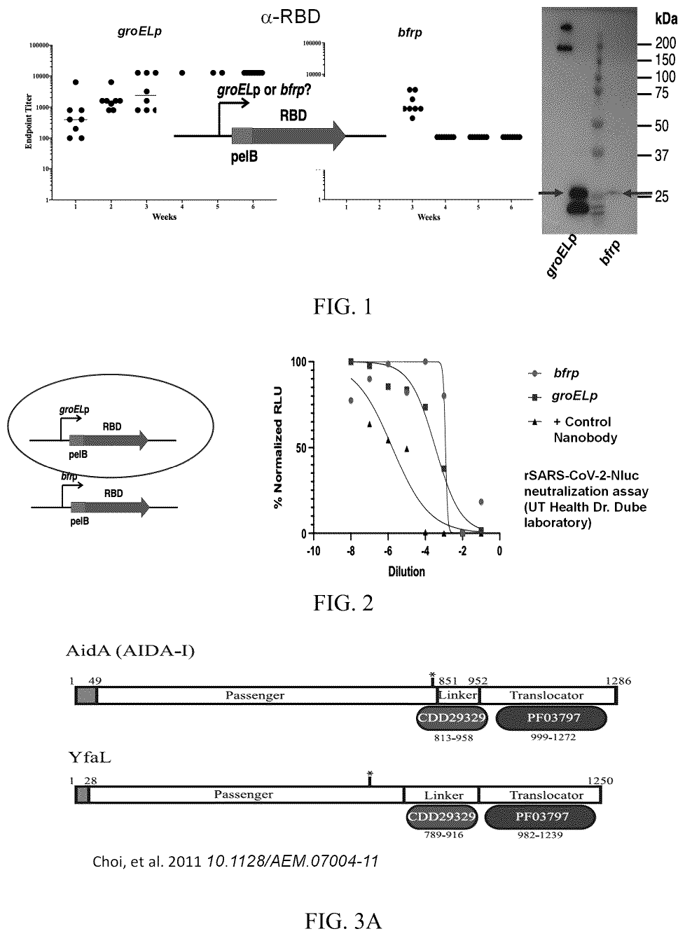

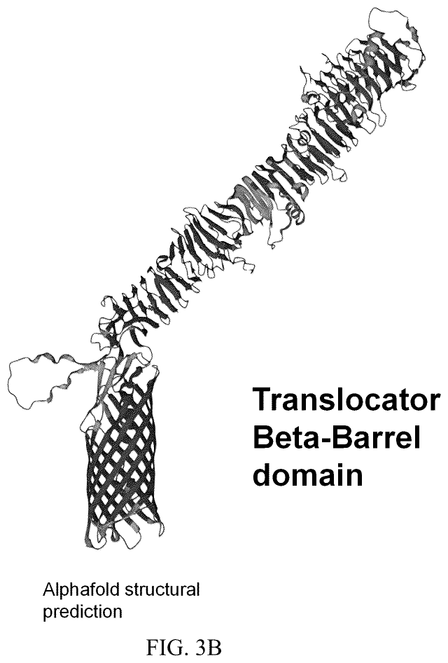

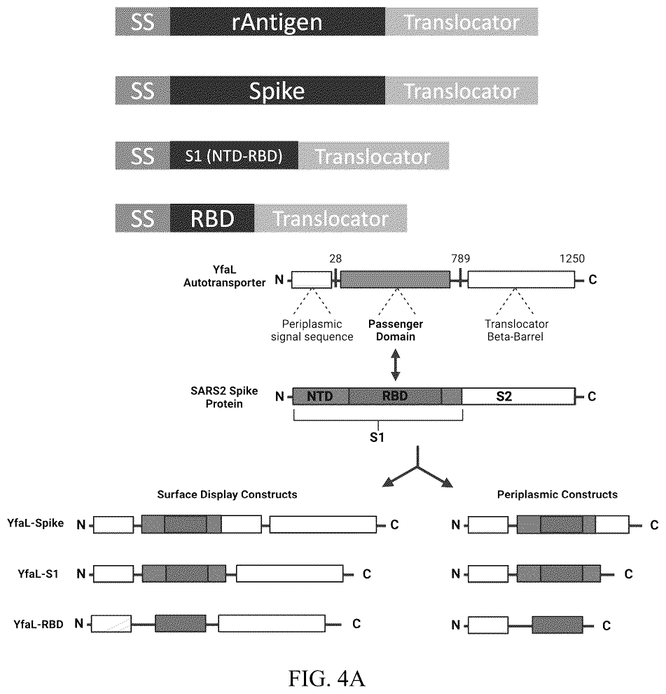

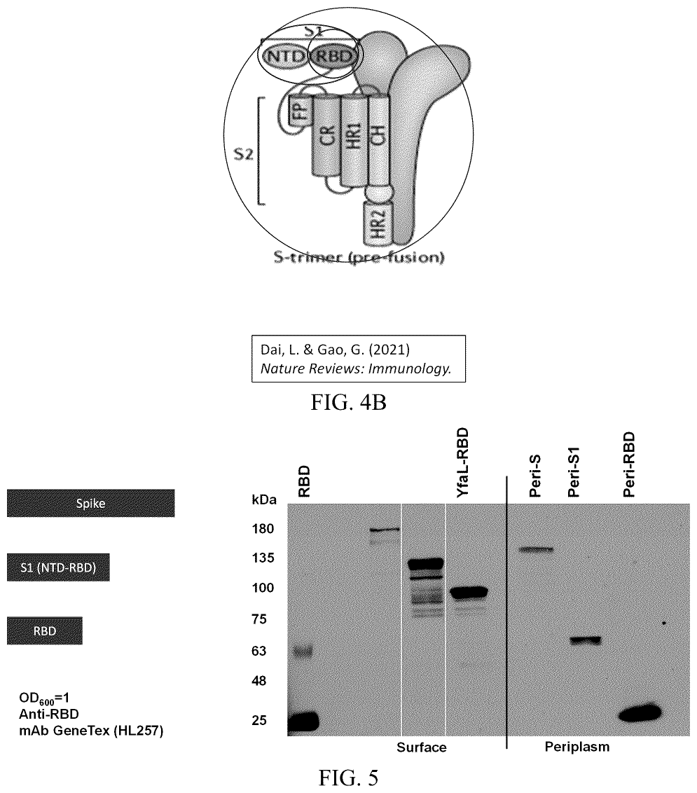

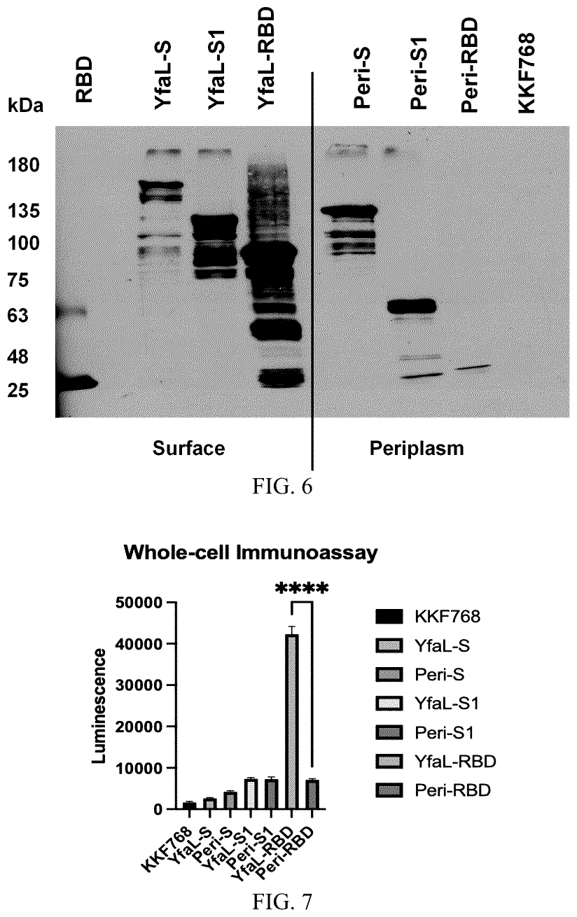

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

Currently, there are no approved vaccines for Francisella tularensis (Ft) or Yersinia pestis (Yp). The vaccine against Bacillus anthracis (Ba) is problematic. The system described herein represents a novel approach that enables vaccination with a single vaccine that can protect against three or more diseases.

Expressing heterologous proteins in Francisella bacteria is challenging for several reasons. Francisella species, particularly Francisella tularensis, are highly pathogenic to humans and require Biosafety Level 3 (BSL-3) containment, which complicates routine laboratory work and limits the number of research facilities equipped to handle them. Compared to more commonly used bacterial expression systems like E. coli, Bacillus subtilis, or Pseudomonas, Francisella has fewer well-developed genetic tools. This includes a limited number of plasmids, promoters, and selection markers that are optimized for Francisella. Francisella has a unique intracellular lifecycle that involves survival within host macrophages. This specificity can affect how heterologous proteins are processed, folded, or modified within the bacterial cell, potentially leading to issues with protein expression or functionality. Differences in codon usage between Francisella and the source organism of the heterologous protein can lead to suboptimal translation efficiency. Some heterologous proteins might be toxic to Francisella, affecting cell viability or leading to selection against cells expressing the protein. The regulatory elements like promoters that work well in other bacteria might not function optimally in Francisella, requiring the use of native or specifically engineered promoters. If the heterologous protein requires specific modifications for activity or stability, Francisella might not provide the necessary machinery, or it might modify the protein in unexpected ways. Francisella has specific growth requirements, including the need for enriched media and often cysteine supplementation, which might not be ideal for all types of protein expression.

### I. Biothreats and Biothreat Vaccines

Embodiments of the invention include compositions and methods for treating a subject infected with or exposed to a pathogen, for example a biothreat. A biothreat refers to harmful biological agents, which can include bacteria, viruses, toxins, and other biological substances capable of causing significant harm to humans, animals, or plants. Biothreats can arise naturally, such as through zoonotic diseases (diseases that jump from animals to humans), accidentally due to laboratory errors or insufficient containment measures, or intentionally through acts of bioterrorism or biological warfare where pathogens or toxins are deliberately released. Intentional use (Bioterrorism/Biological Warfare) involves the deliberate release of a pathogen or toxin (bioweapon or biological weapon) to cause illness or death in people, animals, or plants.

In the U.S., the CDC categorizes biological agents into three groups based on their threat level: Category A poses the highest risk to national security because these agents can be easily disseminated or transmitted from person to person, result in high mortality rates, and might cause public panic or require special action for public health preparedness. Examples of category A agents include Bacillus anthracis (Anthrax), Clostridium botulinum toxin (Botulism), Yersinia pestis (Plague), Variola major (Smallpox) and other pox viruses, Francisella tularensis (Tularemia), Viral hemorrhagic fevers (Filoviruses like Ebola and Marburg, and Arenaviruses like Lassa), and smallpox. Category B is the second highest priority, these agents are moderately easy to disseminate and cause moderate morbidity rates. They include pathogens like salmonella, ricin toxin, and certain viruses causing encephalitis. Category C is emerging threats that could be engineered for mass dissemination because of their availability, ease of production, and potential for high morbidity and mortality. This includes Nipah virus or severe acute respiratory syndrome (SARS). Defense and preparedness against biothreats include developing vaccines, treatments, and detection systems.

Francisella tularensis (F. tularensis) is a Gram-negative coccobacillus with a natural reservoir that includes small mammals such as rabbits, hares, and rodents, as well as aquatic environments and soil. F. tularensis includes at least four subspecies—tularensis (type A), holarctica (type B), novicida, and mediasiatica—and the most virulent of these, F. tularensis subspecies tularensis, can cause infection with doses as low as 10 colony forming units (cfu). Transmission typically occurs by handling of infected animals and carcasses, consumption of contaminated food products, and occasionally through insect vectors. The strain and route of infection determines the progression of the disease, which generally involves spreading to multiple organ systems and the lymphatic system. F. tularensis has been determined to potentially pose a severe threat to human and animal health. Interest in the pathobiology of the bacterium has been rekindled with the recognition that F. tularensis may be deployed as a potent bioweapon due to its ease of dissemination via aerosolization and extremely low infective dose. Left untreated, F. tularensis has the potential to be lethal in 30-60% of infected individuals.

Anthrax is an acute infectious disease caused by the bacterium Bacillus anthracis, primarily affecting animals but also capable of infecting humans. It occurs naturally in soil and commonly affects domestic and wild animals around the world, most frequently in agricultural regions. Transmission to humans typically happens through contact with infected animals or animal products, inhalation of spores, or less commonly through ingestion or skin contact. The disease presents in three main forms in humans: cutaneous (skin), inhalation (lungs), and gastrointestinal, each with varying symptoms and severity. If left untreated, anthrax can be fatal, particularly in its inhalation form, which is also known for its potential use as a biological weapon due to the stability and lethality of its spores.

Yersinia pestis, commonly known as the plague, is a bacterial infection primarily transmitted through the bite of infected fleas, though it can also spread through direct contact with infected animals or through respiratory droplets. Historically infamous for causing pandemics like the Black Death in the 14th century, this disease manifests in three forms: bubonic (affecting the lymph nodes), septicemic (infecting the blood), and pneumonic (infecting the lungs). Each form presents with distinct symptoms, but all are potentially fatal without treatment. Antibiotics can effectively treat the plague if administered early, but without intervention, the bacteria's rapid multiplication can lead to severe tissue damage, organ failure, and death. Today, it remains endemic in certain rodent populations worldwide, though human cases are rare due to modern sanitation and medical advancements.

Certain aspects described herein present a multivalent biothreat vaccine platform for expression of biothreat antigens (e.g., F. tularensis, plague antigen(s) and anthrax antigen(s) using a F. novicida vaccine platform, e.g., F. novicida KKF768. The multivalent vaccine can present 2, 3, 4, 5, 6 or more antigens to 2, 3, 4, 5, 6, or more biothreats or other pathogens. Antigens not naturally expressed by Francisella (heterologous antigens) can be expressed by the vaccine platform using various expression system in the host Francisella bacteria while presenting Francisella antigens as well. As a non-limiting example, an expression vector or expression cassette for a plague antigen (e.g., LcrV) and/or an anthrax antigen (e.g., PA) antigen(s) can be used to express antigens in a live-attenuated Tularemia vaccine, e.g., KKF768, providing a plague/anthrax/tularemia vaccine. Other heterologous antigens can also be expressed in place of or concurrently with the plaque and/or anthrax antigens.

The plasmid expression vectors described herein facilitate the expression of antigens, such as Yp and Ba antigens. The Inventors have demonstrated proof-of-concept with representative antigens Yp LcrV and Ba protective antigens (PA). The vector generates high level antigen expression within live-attenuated Tularemia vaccine strain. LcrV has previously been identified as a protective antigen against Yp. LcrV plays a critical role in the virulence of Yp. It is part of the type III secretion system, which is used by Gram-negative bacteria to inject virulence factors directly into host cells. LcrV itself acts as a multifunctional protein involved in the regulation of secretion of other Yops (Yersinia outer proteins) through this system and directly contributes to the pathogen's ability to evade the host's immune response. Previous work has identified derivatives of the LcrV antigen that increase its immunogenicity. One of these LcrV derivatives is rv10 (see PCT Application PCT/US2005/043779) which was evaluated for use as a subunit vaccine. The Ba PA is the protective antigen that is a component of the anthrax toxin produced by Bacillus anthracis (Ba), the bacterium responsible for anthrax. The PA is one of three protein components of the anthrax toxin, the others being Lethal Factor (LF) and Edema Factor (EF). It is initially synthesized as an inactive precursor, PA83, which has a molecular weight of about 83 kDa. PA has been identified to be the primary protective antigen against Ba, and is in advanced stages of development as a subunit vaccine to protect against Ba. The amino acid sequence used in the design described herein is the native amino acid sequence that occurs in Ba. The Ba antigen is being expressed by the vaccine platform described herein, rather than delivered as a subunit vaccine.

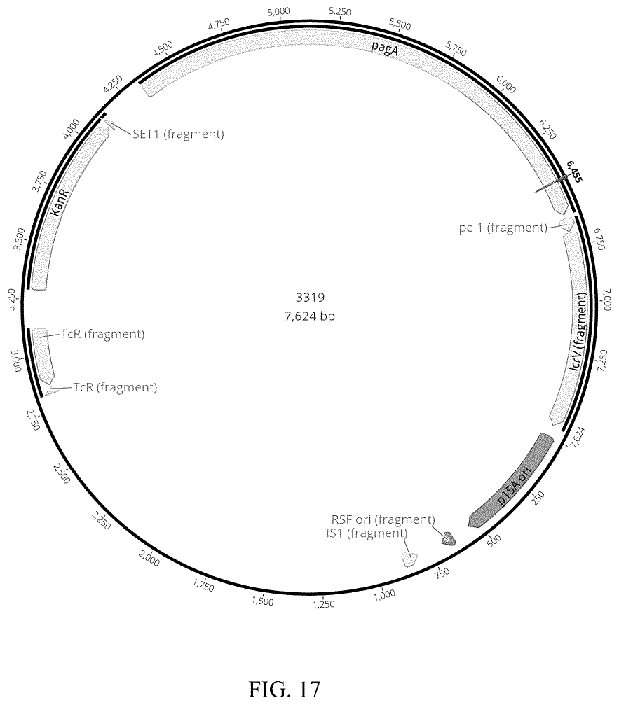

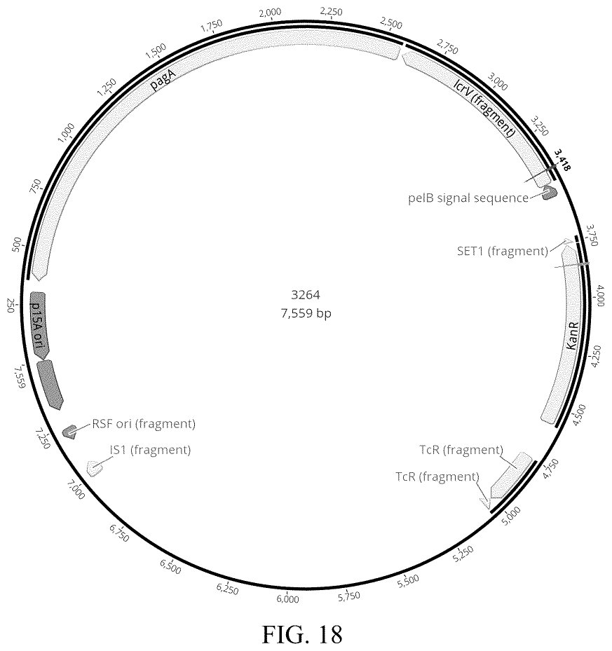

Certain embodiments employ a single vector system that enables high level co-expression of heterologous antigens (e.g., Yp and Ba antigens) within a live attenuated Francisella vaccine. Several variations have been created and described herein which have been engineered for specific subcellular localizations within the Tularemia vaccine strain (periplasmic, cytoplasmic, extracellular). The subcellular localization of these antigens is predicted to affect the immune response to the antigens. The backbone plasmids are new and have been optimized for replication in Francisella and high level antigen expression (see FIG. 17 and FIG. 18 for examples).

Embodiments of the invention include live attenuated Francisella strains expressing heterologous antigens providing for a multivalent vaccine, that is a Francisella vaccine component and 1, 2, 3, 4, 5, 6 or more of a non-Francisella (heterologous) vaccine components.

### A. Antigen Localization Systems and Expression Vectors

The F. tularensis vaccine platform can be engineered to express heterologous antigens. These antigens can be fused to a leader sequence forming a targeted antigen, the presence of the leader results in the processing and localization of the antigen within the bacteria. The antigen can be a full-length protein or an immunogenic portion or fragment of a protein. Antigens can be localized on the surface, in the periplasm, or secreted from the bacterial platform. Leader sequences can include, but are not limited to PelB (MKYLLPTAAAGLLLLAAQPAMH (SEQ ID NO:4))—this sequence is recognized by the bacterial Sec-dependent secretion machinery, allowing the fusion protein to be translocated across the inner membrane; PepO (MKTFILLLFV (SEQ ID NO:5))—this sequence represents the signal peptide that directs the antigen to the Sec-dependent secretion pathway; or TorA (MANNDLFQASRRRFLAQLGGLTVAGMLGPSLLTPRRATAAQAA (SEQ ID NO:6))—this sequence represents the signal peptide that directs the antigen to the Tat-dependent secretion pathway. Antigens can be fused with PelB or PepO leader to direct the fusion protein to the periplasm. Folded antigens can also be fused with a TorA leader to direct the fusion protein to the periplasm in a folded state. Linear peptides or proteins can be fused to a cell surface autotransporter such as YfaL (LSNVTVNGNLTNTSGAVSLONGVAGDTLTVNGDYTGGGTLLLDSELNGDDSVSDQLV MNGNTAGNTTVVVNSITGIGEPTSTGIKVVDFAADPTQFQNNAQFSLAGSGYVNMGAY DYTLVEDNNDWYLRSQEVTPPSPPDPDPTPDPDPTQDPDPTPDPEPTPAYQPVLNAKVG GYLNNLRAANQAFMMERRDHAGGDGQTLNLRVIGGDYHYTAAGQLAQHEDTSTVQL SGDLFSGRWGTDGEWMLGIVGGYSDNQGDSRSSMTGTRADNQNHGYAVGLTSSWFQ HGKQKQGAWLDNWLQYAWFSNDVSEHEDGVDHYHSSGIIASLEAGYQWLPGRGVVIE PQAQVIYQGVQQDDFTAANRARVSQSQGDDIQTRLGLHSEWRTAVHVIPTLDLNYYHD PHSTEIEEDASTISDDA VKQRGEIKVGVTGNISQRVSLRGSVAWQKGSDDFAQTAGFLS MTVKWGT (SEQ ID NO:7; e.g., protein accession NP_418384.1). The YfaL autotransporter protein is a specialized type of protein found in the outer membrane of certain bacteria. YfaL belongs to the autotransporter family, which means it has a unique structure allowing it to transport itself across the bacterial outer membrane. This protein typically features two main domains: a passenger domain and a translocator domain. The passenger domain, which often contains the functional part of the protein, is translocated to the cell surface or secreted, while the translocator domain forms a β-barrel structure embedded in the outer membrane, facilitating this transport.

A targeted antigen can be expressed alone or in combination with 1, 2, 3, 4, 5, 6, or more targeted antigens. Antigens can be encoded in independent expression cassettes producing independent transcripts or multiple antigens can be encoded in the same expression cassette producing a single transcript as monocistronic or a polycistronic message RNA where antigens are processed from a polyprotein or are translated independently, respectively. In certain configurations the antigens (e.g., as a polyprotein) can be further processed into segments (e.g., the protein can contain self-cleavage sites or other protein cleavage sequences).

In one embodiment antigens can be displayed on a Francisella membrane, e.g., periplasm or surface. In one example, KKF768 can be engineered to protect against multiple pathogens. KKF768 has a high safety profile, elicits both humoral and cell-mediated responses, and persists longer than recombinant proteins. In developing KKF768 vaccine platform: immunogenic protective antigen(s) are identified, expression is optimized (promoter identification), and subcellular localization is engineered.

In one example a type V Secretion system is utilized for localization of an antigen. Type V secretion is conserved among Gram-negative bacteria and has been previously used in whole-cell biocatalysis and vaccine development. In certain embodiments antigen presentation and localization is through using an engineered autotransporter, such as YfaL (e.g., NC_002695.2 Escherichia coli O157: H7 str. Sakai; protein accession NP_311143.1). YfaL is a member of the AIDA-I family in E. coli. It has 3 domains: N-terminal signal peptide, passenger domain, and C-terminal linker-translocator. Constructs for periplasmic localization can be generated by removal of the translocator domain

(SEQ ID NO: 9)
(MRVIFLRKEYLSLLPSMIASLESANGVA¹HMRVQPTESIVRFPNI
TNLCPFDEVFNATRFASVYAWNRKRISNCVADYSVLYNLAPFFTF
KCYGVSPTKLNDLCFTNVYADSFVIRGDEVRQIAPGQTGNIADYN
YKLPDDFTGCVIAWNSNKLDSKVSGNYNYLYRLFRKSNLKPFERD
ISTEIYQAGNKPCNGVAGENCYFPLRSYSFRPTYGVGHQPYRVVV
LSFELLHAPATVCGPKKSTNLVKNKCVNF²GTLSNVTVNGNLTNT
SGAVSLQNGVAGDTLTVNGDYTGGGTLLLDSELNGDDSVSDQLVM
NGNTAGNTTVVVNSITGIGEPTSTGIKVVDFAADPTQFQNNAQFS
LAGSGYVNMGAYDYTLVEDNNDWYLRSQEVTPPSPPDPDPTPDPD
PTQDPDPTPDPEPTPAYQPVLNAKVGGYLNNLRAANQAFMMERRD
HAGGDGQTLNLRVIGGDYHYTAAGQLAQHEDTSTVQLSGDLESGR
WGTDGEWMLGIVGGYSDNQGDSRSSMTGTRADNQNHGYAVGLTSS
WFQHGKQKQGAWLDNWLQYAWESNDVSEHEDGVDHYHSSGIIASL
EAGYQWLPGRGVVIEPQAQVIYQGVQQDDFTAANRARVSQSQGDD
IQTRLGLHSEWRTAVHVIPTLDLNYYHDPHSTEIEEDASTISDDA
VKQRGEIKVGVTGNISQRVSLRGSVAWQKGSDDFAQTAGFLSMTV
KW³GT;
1 = YfaL periplasmic localizationsignal sequence,
2 = Omicron RBD,
3 = YfaL Beta-Barrel translocator domain);

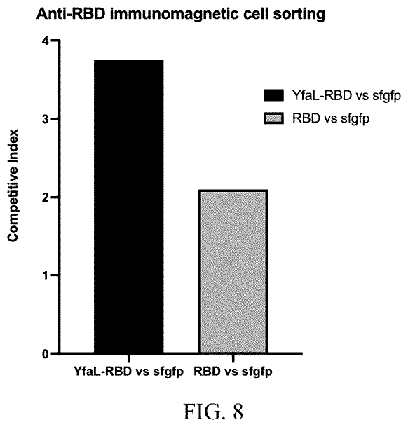

localization on surface of KKF768 can be detected (see FIG. 7). Amino acid sequence for the modified Yfal-RBD (periplasmic) lacking translocator beta-barrel can have thefollowing amino acid sequence: MRVIFLRKEYLSLLPSMIASLFSANGVA¹EFRVQPTESIVRFPNITNLCPFDEVFNATRFAS VYAWNRKRISNCVADYSVLYNLAPFFTFKCYGVSPTKLNDLCFTNVYADSFVIRGDEVR QIAPGQTGNIADYNYKLPDDFTGCVIAWNSNKLDSKVSGNYNYLYRLFRKSNLKPFERD ISTEIYQAGNKPCNGVAGENCYFPLRSYSFRPTYGVGHQPYRVVVLSFELLHAPATVCGP KKSTNLVKNKCVNFGT² (SEQ ID NO:10) 1=Yfal periplasmic localization signal sequence, 2=Omicron RBD (accession: 7WSC_R)). Antigens displayed on the surface of KKF768 will be bound by anti-RBD mAb and emit higher signals than antigens expressed in the periplasm. KKF768/YfaL-RBD light emission was significantly higher than RBD localized to the periplasm, indicating surface display of SARSCOV2RBD on KKF768. FIG. 8 illustrates the detection of YfaL-RBD localization on the surface of KKF768 by magnetic bead-based cell sorting using Anti-rabbit IgG coated, Anti-RBD (GenTex). Mixtures: 1. YfaL-RBD (surface): sfgfp and 2. RBD (intracellular): sfgfp. YfaL-SARSCOV2RBD is detectable on the surface of KKF768.

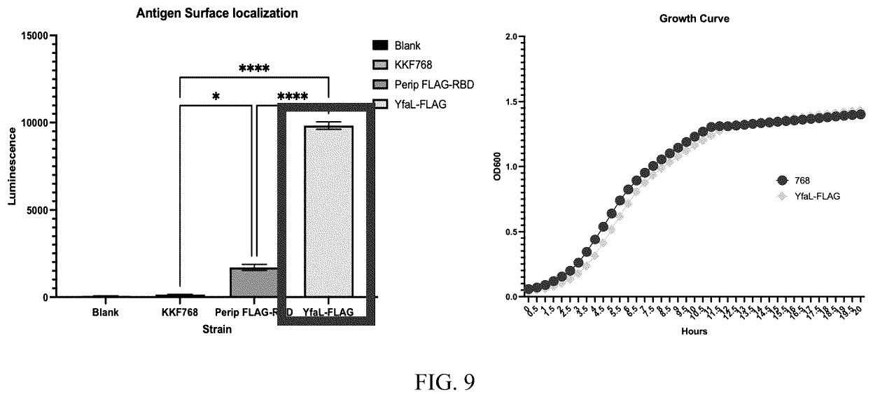

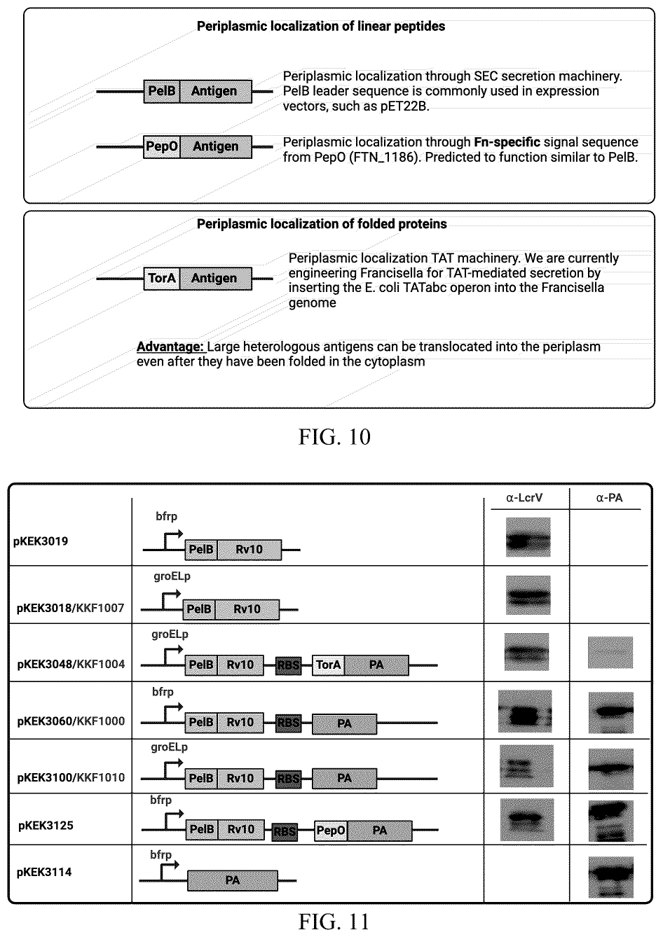

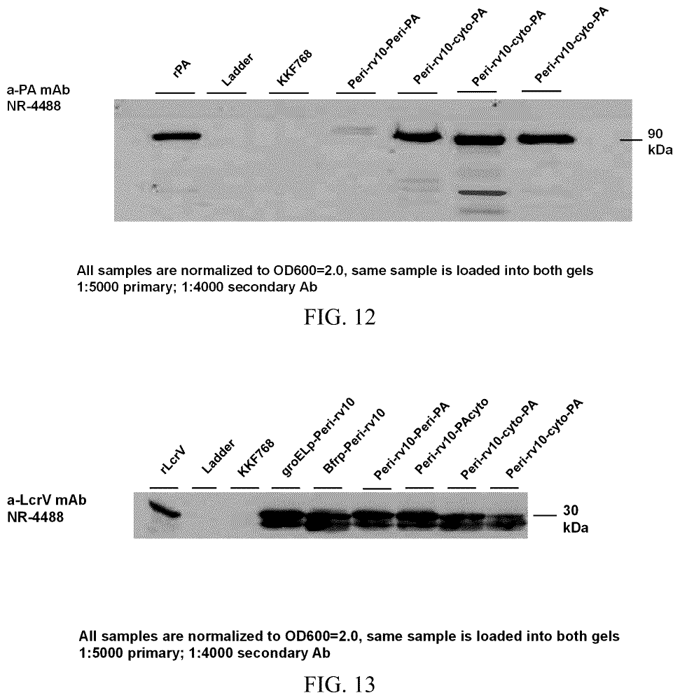

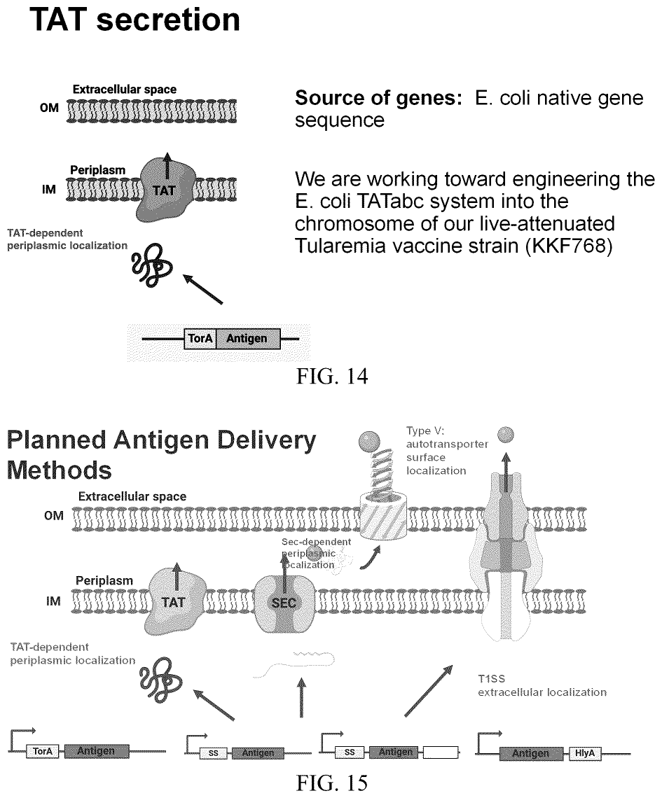

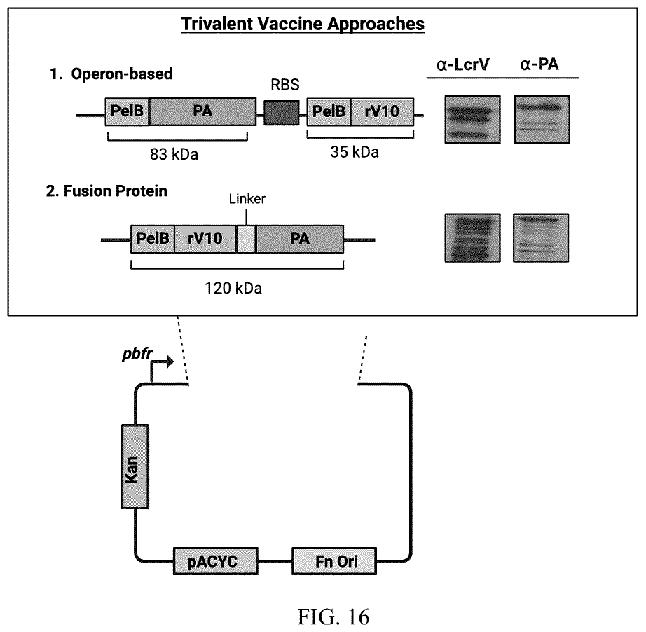

KKF768/YfaL-RBD was shown to induce humoral responses in BALB/c mice Neutralization activity of KKF768 expression of SARS-CoV-2 antigens was assessed in different subcellular localizations (surface vs cytoplasmic vs periplasmic). FIG. 9 illustrates the efficacy of KKF768/YfaL-SARS2 strains in vivo.

Plasmid pKEK3319 (operon-based co-expression of PelB-rV10 and PelB-PA; both localized to periplasm via sec secretion system) within the KKF768 vaccine strain. Plasmid pKEK3319 encodes an operon containing two expression cassettes for PelB-PA and PelB-rV10. pKEK3319 has the nucleic acid sequence of SEQ ID NO:1. The expression vector comprises a native bfr promoter sequence from F. novicida that drives bacterioferritin expression (FTN_1410) in Francisella spp. This promoter has been previously characterized to drive high levels of transcription in Francisella spp,. The expression system contains a ribosome binding site (RBS) that allows each antigen within the operon to be translated independently. An alternate promoter pGroEL has the nucleic acid sequence

(SEQ ID NO: 7)
GACTCAATATCTAGACTTGCAAGAGCTTGGAACTTTGAGATTGTT
CTAAGATGCATACAAATTCAAAATGCTTAAACAAAAATAATTTAA
CAAAGGAGTAAGATTGTTATGAACATTCGTCCATTCTCGACTTTA
GGATACATTCAT

The pKEK3264 plasmid encodes a PelB-rV10-PA fusion which is a fusion of the rV10 and PA antigens localized to the periplasm via PelB leader sequence. pKEK3264 plasmid sequence is provided as SEQ ID NO:2.

### B. Antigens and Vaccines

The present invention further includes methods of using live attenuated F. novicida according to the present invention to vaccinate an animal, including a human being, against antigens and organisms heterologous to F. novicida and to treat an infection or disease. The method comprises administering an effective amount of one or more live attenuated F. novicida strains to the animal such that an immune response to the heterologous antigen is produced in the animal.

Representative antigens include SARS-CoV2 antigen or anthrax or plague antigens. The expression vectors have been engineered for expression and targeted localization of SARS-CoV2 Spike protein antigen, rV10 (Plague) and PA (Anthrax). As a representative example, the rV10 (Plague) and PA (Anthrax) antigens have been engineered into KKF768 (live-attenuated Tularemia vaccine strain) to develop a trivalent vaccine against three biothreats.

The PelB-PA amino acid sequence has the amino acid sequence

(SEQ ID NO: 8)
MKYLLPTAAAGLLLLAAQPAMH¹MEVKQENRLLNESESSSQGLLG
YYFSDLNFQAPMVVTSSTTGDLSIPSSELENIPSENQYFQSAIWS
GFIKVKKSDEYTFATSADNHVTMWVDDQEVINKASNSNKIRLEKG
RLYQIKIQYQRENPTEKGLDFKLYWTDSQNKKEVISSDNLQLPEL
KQKSSNSRKKRSTSAGPTVPDRDNDGIPDSLEVEGYTVDVKNKRT
FLSPWISNIHEKKGLTKYKSSPEKWSTASDPYSDFEKVTGRIDKN
VSPEARHPLVAAYPIVHVDMENIILSKNEDQSTQNTDSQTRTISK
NTSTSRTHTSEVHGNAEVHASFFDIGGSVSAGFSNSNSSTVAIDH
SLSLAGERTWAETMGLNTADTARLNANIRYVNTGTAPIYNVLPTT
SLVLGKNQTLATIKAKENQLSQILAPNNYYPSKNLAPIALNAQDD
FSSTPITMNYNQFLELEKTKQLRLDTDQVYGNIATYNFENGRVRV
DTGSNWSEVLPQIQETTARIIFNGKDLNLVERRIAAVNPSDPLET
TKPDMTLKEALKIAFGFNEPNGNLQYQGKDITEFDFNFDQQTSQN
IKNQLAELNATNIYTVLDKIKLNAKMNILIRDKRFHYDRNNIAVG
ADESVVKEAHREVINSSTEGLLLNIDKDIRKILSGYIVEIEDTEG
LKEVINDRYDMLNISSLRQDGKTFIDFKKYNDKLPLYISNPNYKV
NVYAVTKENTIINPSENGDTSTNGIKKILIFSKKGYEIG²
1 = PelB leader
sequence; 2 = PA antigen.

PelB-rV10 amino acid sequence

(SEQ ID NO: 9)
MKYLLPTAAAGLLLLAAQPAMH¹MIRAYEQNPQHFIEDLEKVR
VEQLTGHGSSVLEELVQLVKDKNIDISIKYDPRKDSEVFANRVIT
DDIELLKKILAYFLPEDAILKGGHYDNQLQNGIKRVKEFLESSPN
TQWELRAFMAVMHFSLTADRIDDDILKVIVDSMNHHGDARSKLRE
ELAELTAELKIYSVIQAEINKHLSSSGTINIHDKSINLMDKNLYG
YTDEEIFKASAEYKILEKMPQTTIQVDGSEKKIVSIKDFLGSENK
RTGALGNLKNSYSYNKDNNELSHFAAIEALNRFIQKYDSVMQRLL
DDTSGK²
1 = PelB leader sequence; 2 = rV10 antigen.

PelB-rV10-PA fusion protein antigen has an amino acid sequence of

(SEQ ID NO: 10)
MKYLLPTAAAGLLLLAAQPAMH¹MIRAYEQNPQHFIEDLEKVRVE
QLTGHGSSVLEELVQLVKDKNIDISIKYDPRKDSEVFANRVITDD
IELLKKILAYFLPEDAILKGGHYDNQLQNGIKRVKEFLESSPNTQ
WELRAFMAVMHFSLTADRIDDDILKVIVDSMNHHGDARSKLREEL
AELTAELKIYSVIQAEINKHLSSSGTINIHDKSINLMDKNLYGYT
DEEIFKASAEYKILEKMPQTTIQVDGSEKKIVSIKDFLGSENKRT
GALGNLKNSYSYNKDNNELSHFAAIEALNRFIQKYDSVMQRLLDD
TSGK²GSGGGGS³EVKQENRLLNESESSSQGLLGYYFSDLNFQAP
MVVTSSTTGDLSIPSSELENIPSENQYFQSAIWSGFIKVKKSDEY
TFATSADNHVTMWVDDQEVINKASNSNKIRLEKGRLYQIKIQYQR
ENPTEKGLDFKLYWTDSQNKKEVISSDNLQLPELKQKSSNSRKKR
STSAGPTVPDRDNDGIPDSLEVEGYTVDVKNKRTFLSPWISNIHE
KKGLTKYKSSPEKWSTASDPYSDFEKVTGRIDKNVSPEARHPLVA
AYPIVHVDMENIILSKNEDQSTQNTDSQTRTISKNTSTSRTHTSE
VHGNAEVHASFFDIGGSVSAGFSNSNSSTVAIDHSLSLAGERTWA
ETMGLNTADTARLNANIRYVNTGTAPIYNVLPTTSLVLGKNQTLA
TIKAKENQLSQILAPNNYYPSKNLAPIALNAQDDFSSTPITMNYN
QFLELEKTKQLRLDTDQVYGNIATYNFENGRVRVDTGSNWSEVLP
QIQETTARIIFNGKDLNLVERRIAAVNPSDPLETTKPDMTLKEAL
KIAFGFNEPNGNLQYQGKDITEFDFNFDQQTSQNIKNQLAELNAT
NIYTVLDKIKLNAKMNILIRDKRFHYDRNNIAVGADESVVKEAHR
EVINSSTEGLLLNIDKDIRKILSGYIVEIEDTEGLKEVINDRYDM
LNISSLRQDGKTFIDFKKYNDKLPLYISNPNYKVNVYAVTKENTI
INPSENGDTSTNGIKKILIFSKKGYEIG⁴
1 = PelB;
2 = rV10 antigen;
3 = Flexible linker sequence;
4 = PA antigen.

### C. Vaccine Compositions

The present invention further includes methods of vaccinating a subject against infection by F. tularensis and a second and/or third biothreat, or treating an F. tularensis infection and infections by a second and/or third organism by administering an effective amount of a pharmaceutical composition comprising one or more live attenuated strains of F. novicida expressing heterologous antigens and a pharmaceutically acceptable carrier. The pharmaceutically acceptable carrier will vary based on the desired method or route of administration and may comprise any suitable liquid, semisolid, or solid known in the art. For example, the carrier may comprise sterile water or saline. The carrier may further comprise, for example, a diluent or other additive necessary to ensure stability of the composition and/or facilitate administration of the composition. Those of ordinary skill in the art will appreciate that there are many additional suitable carriers and compositions that may be used.

The live, attenuated F. novicida strain(s) with or without a carrier may be administered using any suitable method, including, but not limited to, intradermal, intramuscular, intravenous, oral, or intranasal administration, as well as by scarification. In some embodiments, the method may further comprise administering a second dose of the live, attenuated F. novicida strain(s) with or without a carrier at a predetermined time following the initial administration. This booster dose may help to increase the immune response of the animal and provide further protection against or treatment of an infection. In certain aspects the KKF768 strain is used as a live-attenuated F. novicida strain. KKF768 is a Francisella subspecies novicida that is avirulent in humans and has a defined genetic background (attenuating mutation: Δ igID; modifications: ΔOAg^{FN}::OAg^{FTT}).

A heterologous antigen can be a viral or bacterial or cancer antigen. In certain aspects the antigen is a viral antigen. The viral antigen can be SARS virus antigen. The SARS virus antigen can be SARS-CoV2 antigen, such as the SARS virus spike(S) protein. The S protein is an immunodominant antigen during SARS-CoV infection and is the target of effective COVID-19 vaccines. The spike protein S1 subunit determines receptor recognition and contains a receptor-binding domain (RBD) and a N-terminal domain (NTD). The spike protein S2 subunit contains membrane fusion and viral entry domains.

KKF768/SARSCOV2 strains can be used to express variations of SARSCOV2 S protein, e.g., Full-length, S1, RBD. Antigen subcellular localization affects vaccine-induced immunity, e.g., Periplasmic: sec-dependent secretion N-terminal signal sequence, or Surface/extracellular: autotransporter-mediated surface display.

The formulations of the invention are administered in pharmaceutically acceptable solutions, which may routinely contain pharmaceutically acceptable concentrations of salt, buffering agents, preservatives, compatible carriers, and optionally adjuvants and other therapeutic ingredients.

For use in therapy, an effective amount of the live attenuated Francisella as described herein can be administered to a subject by any mode that delivers the live attenuated vaccine to the desired surface. Administering the pharmaceutical composition of the present invention may be accomplished by any means known to the skilled artisan. Routes of administration include but are not limited to oral, parenteral, intramuscular, intranasal, intradermal, sublingual, intratracheal, inhalation, ocular, vaginal, and rectal. Preferred routes are by injection or by inhalation. Vaccine compositions are well known in the pharmaceutical arts.

“Treating” or “treatment” refers to either (i) the prevention of infection or reinfection, e.g., prophylaxis, or (ii) the reduction or elimination of symptoms of the disease of interest, e.g., therapy. “Treating” or “treatment” can refer to the administration of a composition comprising a vaccine prepared and/or stored as described herein. Treating a subject can prevent or reduce the risk of infection and/or induce an immune response to 1, 2, 3, or more pathogens or agents.

Treatment can be prophylactic (e.g., to prevent or delay the onset of the disease, to prevent the manifestation of clinical or subclinical symptoms thereof, or to prevent recurrence of the disease) or therapeutic (e.g., suppression or alleviation of symptoms after the manifestation of the disease). “Preventing” or “prevention” refers to prophylactic administration or vaccination with a vaccine prepared and/or stored as described herein or compositions thereof in a subject who has not been infected or who is symptom-free and/or at risk of infection.

As used herein, the term “immune response” refers to the response of immune system cells to external or internal stimuli (e.g., antigens, cell surface receptors, cytokines, chemokines, and other cells) producing biochemical changes in the immune cells that result in immune cell migration, killing of target cells, phagocytosis, production of antibodies, production of soluble effectors of the immune response, and the like. An “immunogenic” molecule is one that is capable of producing an immune response in a subject after administration.

“Active immunization” refers to the process of administering an antigen(s) (e.g., a vaccine composition or platform described herein) to a subject in order to induce an immune response.

For oral administration, the vaccine composition can be formulated readily by combining the live attenuated vaccine with pharmaceutically acceptable carriers well known in the art. Such carriers enable the vaccine of the invention to be formulated as tablets, pills, dragees, capsules, liquids, gels, syrups, slurries, suspensions and the like, for oral ingestion by a subject to be treated.

For administration by inhalation, the vaccine according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the vaccine and a suitable powder base such as lactose or starch.

In certain embodiments the vaccine composition may be lyophilized. “Lyophilization”, also known as “freeze-drying”, is a process used for preserving biological material such as vaccines, bacteria, and proteins, by removing the water from the sample, which typically involves first freezing the sample and then drying it, under a vacuum, at very low temperatures. Lyophilization methods are well-known in the art. It should be understood that any standard lyophilization procedures known in the art may be used with the compositions and methods provided herein; it is within the capabilities of persons of skill in the art to select procedures for lyophilization. As used herein, the terms “lyophilization medium” and “lyophilization matrix” are used interchangeably to refer to a composition in which samples (e.g., bacteria) are suspended before being subjected to the freeze drying process. Bacteria are generally resuspended in a lyophilization medium prior to lyophilization using standard procedures, as are known in the art. Typically a suitable lyophilization medium for bacteria will help maintain their viability through the freeze drying process and subsequent storage, for example by stabilizing the cells and/or helping to retain structure of biomolecules.

Nasal delivery of a vaccine composition of the present invention is also contemplated. Nasal delivery allows the passage of a pharmaceutical composition of the present invention to the nasal mucosa, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation.

The vaccine, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the vaccine in water-soluble form. Additionally, suspensions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Kits are provided for preventing or treating a plurality of disease states, comprising vaccine composition as described herein. Instructions for use or for carrying out the methods described herein may also be provided in a kit. A kit may further include additional reagents, solvents, buffers, adjuvants, etc., required for carrying out the methods described herein. For example, a kit may comprise a lyophilization medium comprising: mannitol, a disaccharide selected from sucrose, trehalose, and a mixture of sucrose and trehalose; a lyophilization medium.

The technology described herein is not meant to be limited to particular methods, reagents, compounds, compositions or biological systems, which can, of course, vary. It should also be understood that terminology used herein is for the purpose of describing particular aspects only and is not intended to be limiting.

1. A live attenuated Francisella novicida bacterium expressing a heterologous antigen of a second biothreat, the heterologous antigen being operably coupled to a leader sequence or surface protein that localizes the heterologous antigen in the periplasm or on the surface of the live attenuated Francisella novicida bacteria.

2. The live attenuated Francisella novicida bacterium of claim 1, wherein the surface protein is an autotransporter translocator domain.

3. The live attenuated Francisella novicida bacterium of claim 2, wherein the autotransporter is YfaL.

4. The live attenuated Francisella novicida bacterium of claim 1, wherein the leader sequence is selected from PelB, PepO, or TorA.

5. The live attenuated Francisella novicida bacterium of claim 1, wherein two heterologous antigens are expressed as a fusion protein.

6. The live attenuated Francisella novicida bacterium of claim 5, wherein two heterologous antigens expressed as a fusion are separated by a self-cleaving amino acid sequence once translated.

7. The live attenuated Francisella novicida bacterium of claim 5, wherein two heterologous antigens are independently translated from a polycistronic mRNA.

8. The live attenuated Francisella novicida bacterium of claim 1, wherein the second biothreat antigen is a Category A, Category B, and/or Category C antigen.

9. The live attenuated Francisella novicida bacterium of claim 8, wherein the Category A agent is Bacillus anthracis (Anthrax), Clostridium botulinum toxin (Botulism), Yersinia pestis (Plague), pox viruses, Francisella tularensis (Tularemia), Viral hemorrhagic fevers (Filoviruses like Ebola and Marburg, and Arenaviruses like Lassa), or smallpox.

10. The live attenuated Francisella novicida bacterium of claim 1, wherein the second biothreat antigen is from Yersinia pestis (Yp) or Bacillus anthracis (Ba).

11. The live attenuated Francisella novicida bacterium of claim 1, expressing a Yersinia pestis (Yp) antigen and a Bacillus anthracis (Ba) antigen.

12. The live attenuated Francisella novicida bacterium of claim 11, wherein the Yp antigen is LcrV and the Ba antigen is a BA protective antigen (PA).

13. Attenuated Francisella bacterium expressing one or more heterologous antigen, the one or more heterologous antigen being operably coupled to an autotransporter translocator domain.

14. The attenuated bacterium of claim 13, wherein the autotransporter is YfaL.

15. The attenuated bacterium of claim 13, wherein the one or more heterologous antigen is a Category A, Category B, and/or Category C antigen.

16. The attenuated bacterium of claim 15, wherein the Category A agent is Bacillus anthracis, Clostridium botulinum toxin, Yersinia pestis, Francisella tularensis, Viral hemorrhagic fever, or smallpox.

17. The attenuated bacterium of claim 13, wherein the one or more heterologous antigen is a Yersinia pestis (Yp) antigen or Bacillus anthracis (Ba) antigen.

18. The attenuated bacterium of claim 17, wherein the Yp antigen is LcrV and the Ba antigen is a BA protective antigen (PA).

19. The attenuated bacterium of claim 13, wherein the attenuated bacterium is an attenuated Francisella novicida bacterium.

20. The attenuated bacterium of claim 19, wherein the Francisella novicida bacterium is a KKF768 bacterium.


SEQUENCE LISTING (10 sequences)

SEQ ID NO: 1 - DNA, 7625 bp, synthetic construct
  source 1..7625: mol_type = other DNA
atcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcttatggaactgcctcggtgagttttctccttcattacagaaacggctttttcaaaaatatggtattgataatcctgatatgaataaattgcagtttcatttgatgctcgatgagtttttctaaggatccggctgctaacaaagcccgaaaggaagcataatatcatttttattaaaatatctaggttgaattcttagatattttgatagatagataattagatactaaattgataacttataaagaattaaattttcttttgtatgttaacttgattgctaatatgaattatattagttagtatgttgattataatgattagagttttaaataatggagataataatatgaaatacctgctgccgaccgctgctgctggtctgctgctcctcgctgcccagccggcgatgcatatggaagttaaacaggagaaccggttattaaatgaatcagaatcaagttcccaggggttactaggatactattttagtgatttgaattttcaagcacccatggtggttacctcttctactacaggggatttatctattcctagttctgagttagaaaatattccatcggaaaaccaatattttcaatctgctatttggtcaggatttatcaaagttaagaagagtgatgaatatacatttgctacttccgctgataatcatgtaacaatgtgggtagatgaccaagaagtgattaataaagcttctaattctaacaaaatcagattagaaaaaggaagattatatcaaataaaaattcaatatcaacgagaaaatcctactgaaaaaggattggatttcaagttgtactggaccgattctcaaaataaaaaagaagtgatttctagtgataacttacaattgccagaattaaaacaaaaatcttcgaactcaagaaaaaagcgaagtacaagtgctggacctacggttccagaccgtgacaatgatggaatccctgattcattagaggtagaaggatatacggttgatgtcaaaaataaaagaacttttctttcaccatggatttctaatattcatgaaaagaaaggattaaccaaatataaatcatctcctgaaaaatggagcacggcttctgatccgtacagtgatttcgaaaaggttacaggacggattgataagaatgtatcaccagaggcaagacacccccttgtggcagcttatccgattgtacatgtagatatggagaatattattctctcaaaaaatgaggatcaatccacacagaatactgatagtcaaacgagaacaataagtaaaaatacttctacaagtaggacacatactagtgaagtacatggaaatgcagaagtgcatgcgtcgttctttgatattggtgggagtgtatctgcaggatttagtaattcgaattcaagtacggtcgcaattgatcattcactatctctagcaggggaaagaacttgggctgaaacaatgggtttaaataccgctgatacagcaagattaaatgccaatattagatatgtaaatactgggacggctccaatctacaacgtgttaccaacgacttcgttagtgttaggaaaaaatcaaacactcgcgacaattaaagctaaggaaaaccaattaagtcaaatacttgcacctaataattattatccttctaaaaacttggcgccaatcgcattaaatgcacaagacgatttcagttctactccaattacaatgaattacaatcaatttcttgagttagaaaaaacgaaacaattaagattagatacggatcaagtatatgggaatatagcaacatacaattttgaaaatggaagagtgagggtggatacaggctcgaactggagtgaagtgttaccgcaaattcaagaaacaactgcacgtatcatttttaatggaaaagatttaaatctggtagaaaggcggatagcggcggttaatcctagtgatccattagaaacgactaaaccggatatgacattaaaagaagcccttaaaatagcatttggatttaacgaaccgaatggaaacttacaatatcaagggaaagacataaccgaatttgattttaatttcgatcaacaaacatctcaaaatatcaagaatcagttagcggaattaaacgcaactaacatatatactgtattagataaaatcaaattaaatgcaaaaatgaatattttaataagagataaacgttttcattatgatagaaataacatagcagttggggcggatgagtcagtagttaaggaggctcatagagaagtaattaattcgtcaacagagggattattgttaaatattgataaggatataagaaaaatattatcaggttatattgtagaaattgaagatactgaagggcttaaagaagttataaatgacagatatgatatgttgaatatttctagtttacggcaagatggaaaaacatttatagattttaaaaaatataatgataaattaccgttatatataagtaatcccaattataaggtaaatgtatatgctgttactaaagaaaacactattattaatcctagtgagaatggggatactagtaccaacgggatcaagaaaattttaatcttttctaaaaaaggctatgagataggataaaggagaaattaaaaatgaaatacttgttacctactgctgccgcgggcctgctgttgttggccgctcagccggcgatgcatatgattagagcctacgaacaaaacccacaacattttattgaggatctagaaaaagttagggtggaacaacttactggtcatggttcttcagttttagaagaattggttcagttagtcaaagataaaaatatagatatttccattaaatatgatcccagaaaagattcggaggtttttgccaatagagtaattactgatgatatcgaattgctcaagaaaatcctagcttattttctacccgaggatgccattcttaaaggcggtcattatgacaaccaactgcaaaatggcatcaagcgagtaaaagagttccttgaatcatcgccgaatacacaatgggaattgcgggcgttcatggcagtaatgcatttctctttaaccgccgatcgtatcgatgatgatattttgaaagtgattgttgattcaatgaatcatcatggtgatgcccgtagcaagttgcgtgaagaattagctgagcttaccgccgaattaaagatttattcagttattcaagccgaaattaataagcatctgtctagtagtggcaccataaatatccatgataaatccattaatctcatggataaaaatttatatggttatacagatgaagagatttttaaagccagcgcagagtacaaaattctcgagaaaatgcctcaaaccaccattcaggtggatgggagcgagaaaaaaatagtctcgataaaggactttcttggaagtgagaataaaagaaccggggcgttgggtaatctgaaaaactcatactcttataataaagataataatgaattatctcactttgccgctattgaagcactgaaccgtttcattcagaaatatgattcagtgatgcaacgtctgctagatgacacgtctggtaaataatgaactagttctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctagaaatgtaccaaattttacctattttaggatacttttcatgaagctcttctatttttccccagtcctttaatagtctacctttagagtctcgtaaatagttatctttgtgacaggggcctcttttatcttttttaatgtaactatatgttattccaacgtcactattactattatccaaatcttttttagcatgccagtaagaactttcataacttaactctatctttcgacctctttgatatacaacaataaagctatagccagtagtaacaacctgttttacttttgttaaatctattaacttcttatttatttttttatgtttttttgaaaatttaaatatttctatattcattcctacacttcctcaaatccaaatggtagcttatgattctcttctggtttcttttctaatttttttatatttgcaataaaaactctttttctatctttgatttttttattgtcccaattcctccaagtatcatcacaaaccctttcaatatcatgtaaatgatgatgtctaaatattgatctgacataatacagatctaggtctagttcatcacttaacacaacttctctaagtctttcagatgcttcgattggtatgtaatcctctttatttttagtatctaaaagcttttgcttaaattcttcttctgtctctgctaccttactaactgtaaacttgatatttgtaatcttacgaccatgttttctgtgatgatccttgtcatcataggttacaaaaatatccgataattgattaatctcttctagtgctggtaataggaacttatttttaaaatttgaatatctgttgctgtaacttttaggtaaatcaaaatcattaatcatatcatcgacatacaatacgcaatcaactatattagcataccctgcttgttcgcctaatttgcttttgagaagtaagtataatctgcttgaatacttacttttaaatgaaaatagtaactgtctttctgctttagtaaagtactcttgtagttgtatcatgtgtggcattaatgaccaatgaaactcgcaaattaaagcactgcttttagggtctgcttcaatatatgcaaaccagttagctatcttcgtttgttctttattcagccatactggcttagacattattgagtgcattaattgcttcaatctcactctgttatgcttaacccctgtagctttttcaagatcagataggcttatcttatacctgtgaaactctttatcttctcttttaaccattgaggcaactaagaatattaagttttgttcttcttttgtaaggctatactttcctgcaacaagagtattagacatagctatttctttgccagcatttacatttttaacttctttcatagaactagagtcattatctcgatatacaaattctataaaacttctattagtaaaacaactacttcataaaaaaaagtagttttaacgatacaaaaagtagttttaaattcaaaaagtgatacaaaaagtagttttaaattcaaaaagtgatacaaaaagtagttttaaattttttaaaaaagtgcttcaaagccttatgtagcaatacttacagaggattaaaaaaaaatctgacaatatataaagagaatatataaagagaatatcttaggggattttaaaaaaatcccacagactcaaagacttttttgactttttaaatcctagaaactatactttaagtacttatttaagtacatggatttagattatgcaaaccgttaattattcaacttttagaaatgaactatctgattcaatggatagagtaacaaaaaatcatagtcctatgattgtaactagaggttcaaaaaaagaagcagttgttatgatgtcgttagaggattttaaagctttaatgcggtattttatcacagttaaattgctaacgcagtcaggcaccgtgtatgaaatctaacaatgcgctcatcgtcatcctcggcctcgactgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcttttgggttgtcactcatcgtatttggtttataattttaagctaataacctaattataactaattaatagttttgtatcttgaaaaaatagctataaaacttatttaaataacgaagatttttgtgtataaaatatttataacaaaaaaaaggagactaaacacatgagccatattcaacgggaaacgtcttgctccaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtc

SEQ ID NO: 2 - DNA, 7559 bp, synthetic construct
  source 1..7559: mol_type = other DNA
acagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctagaaatgtaccaaattttacctattttaggatacttttcatgaagctcttctatttttccccagtcctttaatagtctacctttagagtctcgtaaatagttatctttgtgacaggggcctcttttatcttttttaatgtaactatatgttattccaacgtcactattactattatccaaatcttttttagcatgccagtaagaactttcataacttaactctatctttcgacctctttgatatacaacaataaagctatagccagtagtaacaacctgttttacttttgttaaatctattaacttcttatttatttttttatgtttttttgaaaatttaaatatttctatattcattcctacacttcctcaaatccaaatggtagcttatgattctcttctggtttcttttctaatttttttatatttgcaataaaaactctttttctatctttgatttttttattgtcccaattcctccaagtatcatcacaaaccctttcaatatcatgtaaatgatgatgtctaaatattgatctgacataatacagatctaggtctagttcatcacttaacacaacttctctaagtctttcagatgcttcgattggtatgtaatcctctttatttttagtatctaaaagcttttgcttaaattcttcttctgtctctgctaccttactaactgtaaacttgatatttgtaatcttacgaccatgttttctgtgatgatccttgtcatcataggttacaaaaatatccgataattgattaatctcttctagtgctggtaataggaacttatttttaaaatttgaatatctgttgctgtaacttttaggtaaatcaaaatcattaatcatatcatcgacatacaatacgcaatcaactatattagcataccctgcttgttcgcctaatttgcttttgagaagtaagtataatctgcttgaatacttacttttaaatgaaaatagtaactgtctttctgctttagtaaagtactcttgtagttgtatcatgtgtggcattaatgaccaatgaaactcgcaaattaaagcactgcttttagggtctgcttcaatatatgcaaaccagttagctatcttcgtttgttctttattcagccatactggcttagacattattgagtgcattaattgcttcaatctcactctgttatgcttaacccctgtagctttttcaagatcagataggcttatcttatacctgtgaaactctttatcttctcttttaaccattgaggcaactaagaatattaagttttgttcttcttttgtaaggctatactttcctgcaacaagagtattagacatagctatttctttgccagcatttacatttttaacttctttcatagaactagagtcattatctcgatatacaaattctataaaacttctattagtaaaacaactacttcataaaaaaaagtagttttaacgatacaaaaagtagttttaaattcaaaaagtgatacaaaaagtagttttaaattcaaaaagtgatacaaaaagtagttttaaattttttaaaaaagtgcttcaaagccttatgtagcaatacttacagaggattaaaaaaaaatctgacaatatataaagagaatatataaagagaatatcttaggggattttaaaaaaatcccacagactcaaagacttttttgactttttaaatcctagaaactatactttaagtacttatttaagtacatggatttagattatgcaaaccgttaattattcaacttttagaaatgaactatctgattcaatggatagagtaacaaaaaatcatagtcctatgattgtaactagaggttcaaaaaaagaagcagttgttatgatgtcgttagaggattttaaagctttaatgcggtattttatcacagttaaattgctaacgcagtcaggcaccgtgtatgaaatctaacaatgcgctcatcgtcatcctcggcctcgactgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcttttgggttgtcactcatcgtatttggtttataattttaagctaataacctaattataactaattaatagttttgtatcttgaaaaaatagctataaaacttatttaaataacgaagatttttgtgtataaaatatttataacaaaaaaaaggagactaaacacatgagccatattcaacgggaaacgtcttgctccaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcttatggaactgcctcggtgagttttctccttcattacagaaacggctttttcaaaaatatggtattgataatcctgatatgaataaattgcagtttcatttgatgctcgatgagtttttctaaggatccggctgctaacaaagcccgaaaggaagcataatatcatttttattaaaatatctaggttgaattcttagatattttgatagatagataattagatactaaattgataacttataaagaattaaattttcttttgtatgttaacttgattgctaatatgaattatattagttagtatgttgattataatgattagagttttaaataatggagataataatatgaaatacctgctgccgaccgctgctgctggtctgctgctcctcgctgcccagccggcgatgcatatgattagagcctacgaacaaaacccacaacattttattgaggatctagaaaaagttagggtggaacaacttactggtcatggttcttcagttttagaagaattggttcagttagtcaaagataaaaatatagatatttccattaaatatgatcccagaaaagattcggaggtttttgccaatagagtaattactgatgatatcgaattgctcaagaaaatcctagcttattttctacccgaggatgccattcttaaaggcggtcattatgacaaccaactgcaaaatggcatcaagcgagtaaaagagttccttgaatcatcgccgaatacacaatgggaattgcgggcgttcatggcagtaatgcatttctctttaaccgccgatcgtatcgatgatgatattttgaaagtgattgttgattcaatgaatcatcatggtgatgcccgtagcaagttgcgtgaagaattagctgagcttaccgccgaattaaagatttattcagttattcaagccgaaattaataagcatctgtctagtagtggcaccataaatatccatgataaatccattaatctcatggataaaaatttatatggttatacagatgaagagatttttaaagccagcgcagagtacaaaattctcgagaaaatgcctcaaaccaccattcaggtggatgggagcgagaaaaaaatagtctcgataaaggactttcttggaagtgagaataaaagaaccggggcgttgggtaatctgaaaaactcatactcttataataaagataataatgaattatctcactttgccgctattgaagcactgaaccgtttcattcagaaatatgattcagtgatgcaacgtctgctagatgacacgtctggtaaaggatccggtggcggagggtctgaagttaaacaggagaaccggttattaaatgaatcagaatcaagttcccaggggttactaggatactattttagtgatttgaattttcaagcacccatggtggttacctcttctactacaggggatttatctattcctagttctgagttagaaaatattccatcggaaaaccaatattttcaatctgctatttggtcaggatttatcaaagttaagaagagtgatgaatatacatttgctacttccgctgataatcatgtaacaatgtgggtagatgaccaagaagtgattaataaagcttctaattctaacaaaatcagattagaaaaaggaagattatatcaaataaaaattcaatatcaacgagaaaatcctactgaaaaaggattggatttcaagttgtactggaccgattctcaaaataaaaaagaagtgatttctagtgataacttacaattgccagaattaaaacaaaaatcttcgaactcaagaaaaaagcgaagtacaagtgctggacctacggttccagaccgtgacaatgatggaatccctgattcattagaggtagaaggatatacggttgatgtcaaaaataaaagaacttttctttcaccatggatttctaatattcatgaaaagaaaggattaaccaaatataaatcatctcctgaaaaatggagcacggcttctgatccgtacagtgatttcgaaaaggttacaggacggattgataagaatgtatcaccagaggcaagacacccccttgtggcagcttatccgattgtacatgtagatatggagaatattattctctcaaaaaatgaggatcaatccacacagaatactgatagtcaaacgagaacaataagtaaaaatacttctacaagtaggacacatactagtgaagtacatggaaatgcagaagtgcatgcgtcgttctttgatattggtgggagtgtatctgcaggatttagtaattcgaattcaagtacggtcgcaattgatcattcactatctctagcaggggaaagaacttgggctgaaacaatgggtttaaataccgctgatacagcaagattaaatgccaatattagatatgtaaatactgggacggctccaatctacaacgtgttaccaacgacttcgttagtgttaggaaaaaatcaaacactcgcgacaattaaagctaaggaaaaccaattaagtcaaatacttgcacctaataattattatccttctaaaaacttggcgccaatcgcattaaatgcacaagacgatttcagttctactccaattacaatgaattacaatcaatttcttgagttagaaaaaacgaaacaattaagattagatacggatcaagtatatgggaatatagcaacatacaattttgaaaatggaagagtgagggtggatacaggctcgaactggagtgaagtgttaccgcaaattcaagaaacaactgcacgtatcatttttaatggaaaagatttaaatctggtagaaaggcggatagcggcggttaatcctagtgatccattagaaacgactaaaccggatatgacattaaaagaagcccttaaaatagcatttggatttaacgaaccgaatggaaacttacaatatcaagggaaagacataaccgaatttgattttaatttcgatcaacaaacatctcaaaatatcaagaatcagttagcggaattaaacgcaactaacatatatactgtattagataaaatcaaattaaatgcaaaaatgaatattttaataagagataaacgttttcattatgatagaaataacatagcagttggggcggatgagtcagtagttaaggaggctcatagagaagtaattaattcgtcaacagagggattattgttaaatattgataaggatataagaaaaatattatcaggttatattgtagaaattgaagatactgaagggcttaaagaagttataaatgacagatatgatatgttgaatatttctagtttacggcaagatggaaaaacatttatagattttaaaaaatataatgataaattaccgttatatataagtaatcccaattataaggtaaatgtatatgctgttactaaagaaaacactattattaatcctagtgagaatggggatactagtaccaacgggatcaagaaaattttaatcttttctaaaaaaggctatgagataggataatgaactagttctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcat

SEQ ID NO: 3 - DNA, 6618 bp, synthetic construct
  source 1..6618: mol_type = other DNA
gtccattctcgactttaggatacattcatatgcgggttatctttctacgcaaggagtatttatctctactcccgtcaatgattgcatctcttttctctgctaacggtgtcgcgcatatgagagtccaaccaacagaatctattgttagatttcctaatattacaaacttgtgcccttttgatgaagtttttaacgccaccagatttgcatctgtttatgcttggaacaggaagagaatcagcaactgtgttgctgattattctgtcctatataatctcgcaccatttttcacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaactctaacaagcttgattctaaggttagtggtaattataattacctgtatagattgtttaggaagtctaatctcaaaccttttgagagagatatttcaactgaaatctatcaggccggtaacaaaccttgtaatggtgttgcaggttttaattgttactttcctttacgatcatatagtttccgacccacttatggtgttggtcaccaaccatacagagtagtagtactttcttttgaacttctacatgcaccagcaactgtttgtggacctaaaaagtctactaatttggttaaaaacaaatgtgtcaatttcggtaccctttccaacgtgacggttaatggcaatctgaccaatacgtccggtgcggttagcctgcaaaatggcgtcgctggcgatacgctgacggtaaacggtgattataccggcggcggtacgctactgctcgatagcgaattaaacggcgatgactcggtaagcgatcaattggtgatgaacggtaatactgctggcaacacaactgtggtggttaactccattacagggattggtgagccgacatcgacaggcattaaagtggttgatttcgcagctgatcccacgcagtttcaaaacaatgcgcagttcagtctggcaggcagcggctacgtcaatatgggagcgtatgactacacgctggtggaagataacaacgactggtatctgcgatcgcaagaagtaacgccgccatcgccacctgatccagacccgactcccgatcctgatcccacgcaggatcctgatccaacacccgacccggaacctacgcctgcttaccagccggtgttgaatgccaaagttggcggttatctcaataacctgcgggcggcaaatcaggcgtttatgatggagcgacgcgatcacgcaggtggcgatggtcagacgctgaatttacgtgttatcggcggagattatcattacacagcagcggggcaactggctcaacatgaagacacttctacggtgcaacttagcggcgatctgtttagcgggcgctggggcacggatggcgagtggatgcttgggattgttggtggctacagcgataaccagggcgacagccgctcgagtatgaccggaactcgcgccgataaccagaaccacggttatgcggttgggctgacctcaagctggtttcagcacggtaagcagaagcagggggcctggctggataactggttgcagtacgcgtggtttagcaatgatgtttctgaacatgaagatggcgtggatcattaccattcgtcggggattatcgcctcgctggaagcggggtatcagtggttaccggggcgtggtgtggtgattgaaccgcaggcgcaggtgatttatcagggcgtgcagcaggatgattttaccgccgctaaccgtgcgcgcgtgtcacaatcgcagggtgatgatattcagacgcggctgggtttacacagcgaatggcgtaccgctgttcatgtcataccaacattagatctgaattattatcacgatccccattcgacggaaattgaagaagatgccagcactatcagtgacgatgcggtgaagcaacggggtgaaataaaagtgggagtcacgggcaatatcagtcagcgagtttcgctgcgtggtagcgtggcgtggcagaaagggagtgatgattttgcccagacggcagggtttttgtcgatgacggtgaaatggggtacctaaactagttctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctagaaatgtaccaaattttacctattttaggatacttttcatgaagctcttctatttttccccagtcctttaatagtctacctttagagtctcgtaaatagttatctttgtgacaggggcctcttttatcttttttaatgtaactatatgttattccaacgtcactattactattatccaaatcttttttagcatgccagtaagaactttcataacttaactctatctttcgacctctttgatatacaacaataaagctatagccagtagtaacaacctgttttacttttgttaaatctattaacttcttatttatttttttatgtttttttgaaaatttaaatatttctatattcattcctacacttcctcaaatccaaatggtagcttatgattctcttctggtttcttttctaatttttttatatttgcaataaaaactctttttctatctttgatttttttattgtcccaattcctccaagtatcatcacaaaccctttcaatatcatgtaaatgatgatgtctaaatattgatctgacataatacagatctaggtctagttcatcacttaacacaacttctctaagtctttcagatgcttcgattggtatgtaatcctctttatttttagtatctaaaagcttttgcttaaattcttcttctgtctctgctaccttactaactgtaaacttgatatttgtaatcttacgaccatgttttctgtgatgatccttgtcatcataggttacaaaaatatccgataattgattaatctcttctagtgctggtaataggaacttatttttaaaatttgaatatctgttgctgtaacttttaggtaaatcaaaatcattaatcatatcatcgacatacaatacgcaatcaactatattagcataccctgcttgttcgcctaatttgcttttgagaagtaagtataatctgcttgaatacttacttttaaatgaaaatagtaactgtctttctgctttagtaaagtactcttgtagttgtatcatgtgtggcattaatgaccaatgaaactcgcaaattaaagcactgcttttagggtctgcttcaatatatgcaaaccagttagctatcttcgtttgttctttattcagccatactggcttagacattattgagtgcattaattgcttcaatctcactctgttatgcttaacccctgtagctttttcagaatcagataggcttatcttatacctgtgaaactctttatcttctcttttaaccattgaggcaactaagaatattaagttttgttcttcttttgtaaggctatactttcctgcaacaagagtattagacatagctatttctttgccagcatttacatttttaacttctttcatagaactagagtcattatctcgatatacaaattctataaaacttctattagtaaaacaactacttcataaaaaaaagtagttttaacgatacaaaaagtagttttaaattcaaaaagtgatacaaaaagtagttttaaattcaaaaagtgatacaaaaagtagttttaaattttttaaaaaagtgcttcaaagccttatgtagcaatacttacagaggattaaaaaaaaatctgacaatatataaagagaatatataaagagaatatcttaggggattttaaaaaaatcccacagactcaaagacttttttgactttttaaatcctagaaactatactttaagtacttatttaagtacatggatttagattatgcaaaccgttaattattcaacttttagaaatgaactatctgattcaatggatagagtaacaaaaaatcatagtcctatgattgtaactagaggttcaaaaaaagaagcagttgttatgatgtcgttagaggattttaaagctttaatgcggtattttatcacagttaaattgctaacgcagtcaggcaccgtgtatgaaatctaacaatgcgctcatcgtcatcctcggcctcgactgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcttttgggttgtcactcatcgtatttggtttataattttaagctaataacctaattataactaattaatagttttgtatcttgaaaaaatagctataaaacttatttaaataacgaagatttttgtgtataaaatatttataacaaaaaaaaggagactaaacacatgagccatattcaacgggaaacgtcttgctccaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaggatccggctgctaacaaagcccgaaaggaagcggccgctgaaaaataaacttaattattatatatgttatttagctagtttttttaattaaagttaaaatcgagagcttgtttgacaaaaaaacaaaaaaatttcttgaaaattttttttttgactcaatatctagacttgcaagagcttggaactttgagattgttctaagatgcatacaaattcaaaatgcttaaacaaaaataatttaacaaaggagtaagattgttatgaacattc

SEQ ID NO: 4 - protein, 22 aa, synthetic construct
MKYLLPTAAAGLLLLAAQPAMH

SEQ ID NO: 5 - protein, 10 aa, synthetic construct
MKTFILLLFV

SEQ ID NO: 6 - protein, 43 aa, synthetic construct
MANNDLFQASRRRFLAQLGGLTVAGMLGPSLLTPRRATAAQAA

SEQ ID NO: 7 - protein, 467 aa, synthetic construct
LSNVTVNGNLTNTSGAVSLQNGVAGDTLTVNGDYTGGGTLLLDSELNGDDSVSDQLVMNGNTAGNTTVVVNSITGIGEPTSTGIKVVDFAADPTQFQNNAQFSLAGSGYVNMGAYDYTLVEDNNDWYLRSQEVTPPSPPDPDPTPDPDPTQDPDPTPDPEPTPAYQPVLNAKVGGYLNNLRAANQAFMMERRDHAGGDGQTLNLRVIGGDYHYTAAGQLAQHEDTSTVQLSGDLFSGRWGTDGEWMLGIVGGYSDNQGDSRSSMTGTRADNQNHGYAVGLTSSWFQHGKQKQGAWLDNWLQYAWFSNDVSEHEDGVDHYHSSGIIASLEAGYQWLPGRGVVIEPQAQVIYQGVQQDDFTAANRARVSQSQGDDIQTRLGLHSEWRTAVHVIPTLDLNYYHDPHSTEIEEDASTISDDAVKQRGEIKVGVTGNISQRVSLRGSVAWQKGSDDFAQTAGFLSMTVKWGT

SEQ ID NO: 8 - protein, 255 aa, synthetic construct
MRVIFLRKEYLSLLPSMIASLFSANGVAHMRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFGT

SEQ ID NO: 9 - protein, 722 aa, synthetic construct
MRVIFLRKEYLSLLPSMIASLFSANGVAHMRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFGTLSNVTVNGNLTNTSGAVSLQNGVAGDTLTVNGDYTGGGTLLLDSELNGDDSVSDQLVMNGNTAGNTTVVVNSITGIGEPTSTGIKVVDFAADPTQFQNNAQFSLAGSGYVNMGAYDYTLVEDNNDWYLRSQEVTPPSPPDPDPTPDPDPTQDPDPTPDPEPTPAYQPVLNAKVGGYLNNLRAANQAFMMERRDHAGGDGQTLNLRVIGGDYHYTAAGQLAQHEDTSTVQLSGDLFSGRWGTDGEWMLGIVGGYSDNQGDSRSSMTGTRADNQNHGYAVGLTSSWFQHGKQKQGAWLDNWLQYAWFSNDVSEHEDGVDHYHSSGIIASLEAGYQWLPGRGVVIEPQAQVIYQGVQQDDFTAANRARVSQSQGDDIQTRLGLHSEWRTAVHVIPTLDLNYYHDPHSTEIEEDASTISDDAVKQRGEIKVGVTGNISQRVSLRGSVAWQKGSDDFAQTAGFLSMTVKWGT

SEQ ID NO: 10 - protein, 255 aa, synthetic construct
MRVIFLRKEYLSLLPSMIASLFSANGVAEFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFGT